(19) United States
(12) Patent Application Publication
(10) Pub. No.: US 2026/0295124 A1
(43) Pub. Date: Oct. 1, 2026

(54) COMPOSITION FOR PREVENTING OR TREATING CAPSULAR CONTRACTURE CAUSED BY IMPLANT SURGERY AND USE THEREOF

(71) Applicant: SOONCHUNHYANG UNIVERSITY INDUSTRY ACADEMY COOPERATION FOUNDATION, Asan-si (KR)

(72) Inventors: Jungkyun IM, Asan-si (KR); Mohuya PAUL, Asan-si (KR); Woo jin SONG, Gwacheon-si (KR)

(73) Assignee: SOONCHUNHYANG UNIVERSITY INDUSTRY ACADEMY COOPERATION FOUNDATION, Asan-si (KR)

(21) Appl. No.: 19/414,590

(22) Filed: Dec. 10, 2025

(30) Foreign Application Priority Data
Mar. 25, 2025 (KR) 10-2025-0038083

(51) Int. Cl. A61L 27/44; A61F 2/12; A61L 27/54; D04H 1/4291; D04H 1/4309; D04H 1/435; D04H 1/728

(52) U.S. Cl. CPC A61L 27/44; A61F 2/12; A61L 27/54; D04H 1/4291; D04H 1/4309; D04H 1/435; D04H 1/728; A61L 2300/22; A61L 2300/436; A61L 2400/02

(57) ABSTRACT

The present disclosure relates to PCL electrospun fibers comprising fish oil and montelukast sodium, a method for preparing the same, and use thereof. According to the present disclosure, the electrospun fibers comprising fish oil and montelukast sodium may significantly reduce collagen over-deposition or fibrosis over-production when implanted into an animal model, inhibit the activation of myofibroblasts, develop fibrosis of the implanted surrounding tissue to a late stage, formation, suppress inhibit bacterial adhesion and biofilm inflammatory cytokines, and stabilize a tissue structure by balancing tissue repair and fibrosis, thereby significantly treating capsular contracture. Therefore, the present disclosure may be effectively used for the prevention, improvement, or treatment of capsular contracture.

### CROSS-REFERENCE TO RELATED APPLICATIONS

This application claims the priority of Korean Patent Application No. 10-2025-0038083 filed on Mar. 25, 2025, in the Korean Intellectual Property Office, the disclosure of which is incorporated herein by reference.

### INCORPORATION BY REFERENCE OF SEQUENCE LISTING

The content of the electronically submitted sequence listing, file name: Q314859 Sequence Listing as filed; size: 16, 771 bytes; and date of creation: Dec. 9 2025, filed herewith, is incorporated herein by reference in its entirety.

### BACKGROUND

### Field

The present disclosure relates to a composition for preventing or treating capsular contracture caused by implant surgery and use thereof.

### Description of the Related Art

Recently, as life expectancy after treatment of breast-related diseases such as breast cancer has increased and the cosmetic aspect becomes more emphasized, various methods for breast reconstruction after mastectomy have been commonly used. As an example of these breast reconstruction methods, there has been used breast reconstruction that first inserts a tissue expander to expand soft tissue to a level suitable for implant a prosthesis, and then second replaces the tissue expander with a permanent prosthesis.

However, since the tissue expander or prosthesis is a foreign substance during breast reconstruction, capsular contracture may occur during implantation. The capsular contracture is a symptom in which a thick film is formed around the inserted tissue expander or prosthesis to be harder to the touch, thereby accompanying inflammation and the like. The capsular contracture is considered to be caused by bacterial contamination that forms a biofilm around the implanted implant and an inflammatory response related to a silicone implant, which is the implanted prosthesis.

The secretion of inflammatory cytokines such as interleukin-6 (IL-6), IL-1β, IL-8, IL-4, IL-1β, interferon-gamma (IFN-γ), and tumor necrosis factor-alpha (TNF-α) activates M1 and M2 macrophages around the breast tissues. These macrophages sequentially stimulate the fibrosis process, and as the fibrosis process begins, thick scar tissue begins to form around the silicone implant as a failure of the wound healing process. Fibroblasts associated with the fibrosis process continue to migrate to the ‘wound site’ and deposit collagen and other extracellular matrixes to form a new tissue scaffold. Such excess tissue deposition results in the formation of a thick layer of scar tissue around the silicone implant, and the formed fibrillar tissues exert pressure on the surrounding breast tissue to cause pain and discomfort. As the capsular contracture advances, the pressure intensifies, and the capsular contracture requires multiple correction surgeries until the symptoms disappear. These surgeries include partial or complete capsulectomy and implant removal or replacement. However, surgical procedures have disadvantages, such as infection, aesthetic complications, and even recurrence of capsular contracture. It is reported that capsular contracture occurs in approximately 19 to 25% of patients undergoing breast reconstruction surgeries, often leading to reoperation, and thus, effective prevention strategies are needed.

Meanwhile, over the last decade, electrospun (e-spun) fibers have been widely used in the biomedical field due to their excellent properties. The electrospinning process itself: versatile, fast, and efficient, thereby preparing e-spun fibers industrially advantageously. The e-spun fibers may be prepared variously by changing electrospinning parameters, such as applied voltage, solution flow rate, and a distance between a needle tip and a collector. Due to unique nature and fine tunability, the e-spun fibers may improve biocompatibility and enhance therapeutic efficacy, and particularly, are effectively used for poorly water-soluble drugs.

Therefore, there is a need for the development of a drug delivery system capable of continuously delivering drugs to prevent and treat capsular contracture using the e-spun fibers.

### SUMMARY

An object of the present disclosure is to provide electrospun fibers.

Another object of the present disclosure is to provide a pharmaceutical composition for preventing or treating capsular contracture.

Yet another object of the present disclosure is to provide a quasi-drug composition for preventing or improving capsular contracture.

Still another object of the present disclosure is to provide a composition for implantation.

Still another object of the present disclosure is to provide a method for preventing or treating capsular contracture in a subject other than a human.

Still another object of the present disclosure is to provide a prosthesis.

Still another object of the present disclosure is to provide a method for preparing electrospun fibers.

Still another object of the present disclosure is to provide a method for preparing a breast implant composition.

In order to achieve the object, the present disclosure provides an electrospun fiber including fish oil and a leukotriene receptor antagonist.

In order to achieve another object, the present disclosure provides a pharmaceutical composition for preventing or treating capsular contracture, including electrospun fibers containing fish oil and a leukotriene receptor antagonist.

In order to achieve another object, the present disclosure provides a quasi-drug composition for preventing or improving capsular contracture, including electrospun fibers containing fish oil and a leukotriene receptor antagonist.

In order to achieve another object, the present disclosure provides a composition for implantation including electrospun fibers containing fish oil and a leukotriene receptor antagonist.

In order to achieve another object, the present disclosure provides a method for preventing or treating capsular contracture in a subject other than a human, including implanting a composition for implantation, including electrospun fibers containing fish oil and a leukotriene receptor antagonist, into a subject other than a human.

In order to achieve another object, the present disclosure provides a prosthesis including electrospun fibers containing fish oil and a leukotriene receptor antagonist; and a biocompatible structure.

In order to achieve another object, the present disclosure provides a method for preparing electrospun fibers containing fish oil and a leukotriene receptor antagonist, including dissolving electrospun fibers in a solvent; preparing a first mixture by mixing fish oil into the solvent in which the electrospun fibers are dissolved; preparing a second mixture by adding a leukotriene receptor antagonist to the first mixture; and spinning the second mixture using an electrospinner.

In order to achieve another object, the present disclosure provides a method for preparing a breast implant composition including: dissolving electrospun fibers in a solvent; preparing a first mixture by mixing fish oil into the solvent in which the electrospun fibers are dissolved; preparing a second mixture by adding a leukotriene receptor antagonist to the first mixture; spinning the second mixture using an electrospinner; and coating the surface of a biocompatible structure with the spun electrospun fibers.

According to the present disclosure, electrospun fibers containing fish oil and montelukast sodium can significantly reduce collagen over-deposition or fibrosis over-production when implanted into an animal model, inhibit the activation of myofibroblasts, develop fibrosis of the implanted surrounding tissue to a late stage, inhibit bacterial adhesion and biofilm formation, inhibit inflammatory cytokines, and stabilize a tissue structure by balancing repair and fibrosis, thereby tissue significantly treating capsular contracture. Therefore, the present disclosure can be effectively used for the prevention, alleviation, or treatment of capsular contracture.

The effects of the present disclosure are not limited to the aforementioned effects, and other effects, which are not mentioned above, will be apparently understood to a person having ordinary skill in the art from the following description.

The objects to be achieved by the present disclosure, the means for achieving the objects, and the effects of the present disclosure described above do not specify essential features of the claims, and, thus, the scope of the claims is not limited to the disclosure of the present disclosure.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 is a schematic diagram illustrating a method for preparing electrospun fibers using fish oil, montelukast sodium (MTKS), and PCL fibers according to the present disclosure, an implant using the same, and an effect of implantation into an animal model thereof;

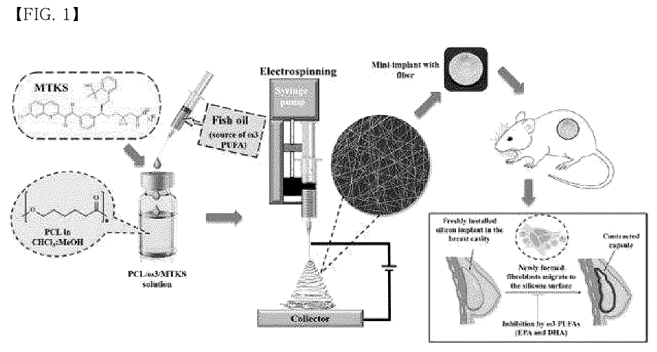

FIG. 2 shows results of confirming an optimal PCL concentration in electrospun fibers of the present disclosure. (A) of FIG. 2 shows results of confirming the morphology of electrospun fibers using 10% PCL, (B) of FIG. 2 shows results of confirming the morphology of electrospun fibers using 12% PCL, (C) of FIG. 2 shows results of confirming the morphology of electrospun fibers using 15% PCL, (D) of FIG. 2 shows results of confirming the average diameter of the electrospun fibers using 10% PCL, (E) of FIG. 2 shows results of confirming the average diameter of the electrospun fibers using 12% PCL, and (F) of FIG. 2 shows results of confirming the average diameter of the electrospun fibers using 15% PCL;

FIG. 3 shows results of confirming an optimal fish oil concentration in electrospun fibers of the present disclosure;

FIG. 4 shows results of FT-IR analysis of PCL fiber, fish oil, MTKS, fibers containing PCL and fish oil (PCL-ω3 fiber), and electrospun fibers according to the present disclosure (PCL-ω3-MTKS fiber);

FIG. 5 shows results of confirming the morphology of the electrospun fibers (PCL-ω3-MTKS fiber) according to the present disclosure. (A) of FIG. 5 shows a result of confirming the morphology of the PCL fiber, (B) of FIG. 5 shows a result of confirming the morphology of the PCL-ω3 fiber, (c) of FIG. 5 shows a result of confirming the morphology of the PCL-ω3-MTKS fiber, (D) of FIG. 5 shows a result of confirming distributions of diameters of the PCL fiber, (E) of FIG. 5 shows a result of confirming distributions of diameters of the PCL-ω3 fiber, and (F) of FIG. 5 shows a result of confirming distributions of diameters of the PCL-ω3-MTKS fiber;

FIG. 6 shows results of confirming the hydrophilicity of the electrospun fibers (PCL-ω3-MTKS fiber) according to the present disclosure. (A) of FIG. 6 shows a result of confirming a water droplet, in which (A) (i) of FIG. 6 shows a result of PCL fiber, (A) (ii) of FIG. 6 shows a result of PCL-ω3 fiber, (A) (iii) of FIG. 6 shows a result of PCL-ω3-MTKS fiber, and (A) (iv) of FIG. 6 shows a result of a water contact angle of each fiber. (B) of FIG. 6 shows a result of comparing XRD spectra of as-spun fibers and solid precursors;

FIG. 7 shows results of confirming thermal stability of the electrospun fibers (PCL-ω3-MTKS fiber) according to the present disclosure. (A) of FIG. 7 shows a result of thermogravimetric analysis (TGA), and (B) of FIG. 7 shows a result of derivatives of the TGA curve (DTG, %/° C.);

FIG. 8 shows results of confirming selective removal of fish oil by washing the electrospun fibers (PCL-ω3-MTKS fiber) according to the present disclosure with hexane. (A) of FIG. 8 is a result of comparing FT-IR spectra before and after washing with hexane, (B) of FIG. 8 is an image of confirming the morphology of the PCL fiber before washing, (C) of FIG. 8 is an image of confirming the morphology of the PCL-ω3-MTKS fiber before washing, (D) of FIG. 8 is an image of confirming the morphology of the PCL fiber after washing, and (E) of FIG. 8 is an image of confirming the morphology of the CL-ω3-MTKS fiber;

FIG. 9 shows results of confirming the drug encapsulation efficiency of the electrospun fibers (PCL-ω3-MTKS fiber) according to the present disclosure. (A) of FIG. 9 shows results of confirming the release of fish oil contained in the fibers over time, and (B) of FIG. 9 shows results of confirming the release of MTKS contained in the fibers over time;

FIG. 10 shows results of confirming the formation of capsular contracture by fabricating a silicone prosthesis by coating the surface with electrospun fibers (PCL-ω3-MTKS fiber) according to the present disclosure and implanting the silicone prosthesis into an animal model. (A) of FIG. 10 is an image of a silicone prosthesis surface-coated with the prepared PCL-ω3-MTKS fiber, (B) and (C) of FIG. 10 are images of the silicone prosthesis inserted into an animal model, and (D) of FIG. 10 is an image of confirming the silicone prosthesis surface-coated with PCL-ω3-MTKS fiber obtained from an animal model after 90 days and surrounding tissues thereof;

FIG. 11 shows results of confirming the formation of capsular contracture in a surrounding tissue of the silicone prosthesis surface-coated with PCL-ω3-MTKS fiber obtained from an animal model. (A) of FIG. 11 shows results of histoimmunological staining, and (B) of of FIG. 11 shows results of confirming a capsule thickness of each experimental group;

FIG. 12 shows results of confirming the formation of capsular contracture in a surrounding tissue of the silicone prosthesis surface-coated with PCL-ω3-MTKS fiber obtained from an animal model. (A) of FIG. 12 shows results of confirming collagen and muscle fibers, and (B) of FIG. 12 shows results of confirming the expression of α-SMA, a key protein of myofibroblasts;

FIG. 13 shows results 4 confirming and comparing the expression of inflammatory cytokines and fibrosis-related factors in a surrounding tissue of the silicone prosthesis surface-coated with PCL-ω3-MTKS fiber obtained from an animal model; and

FIG. 14 shows results of evaluating the degree of fibrosis in a surrounding tissue of the silicone prosthesis surface-coated with PCL-ω3-MTKS fiber obtained from an animal model. (A) to (D) images and corresponding collagen fiber of FIG. 14 are SHG orientation histogram plot results of the tissues around a silicone prosthesis surface-coated with PCL-ω3-MTKS fiber obtained from an animal model, (E) of FIG. 14 is a collagen fiber angle boxplot result, (F) of FIG. 14 is a correlation analysis result of fibrillar collagen, and (G) of FIG. 14 is an entropy comparison result of a collagen orientation angle between experimental groups.

### DETAILED DESCRIPTION OF THE EMBODIMENT

Hereinafter, the exemplary embodiment of the present disclosure will be described with reference to the accompanying drawings and exemplary embodiments as follows. Scales of components illustrated in the accompanying drawings are different from the real scales for the purpose of description, so that the scales are not limited to those illustrated in the drawings.

Hereinafter, the present disclosure will be described in detail.

The present disclosure provides electrospun fibers including fish oil and a leukotriene receptor antagonist.

In the present disclosure, the electrospun fibers may be at least one selected from the group consisting of poly(ε-caprolactone) (PCL), polyvinyl alcohol (PVA), polystyrene (PS), poly(lactic-co-glycolic) acid (PLGA), and poly(l-lactic acid) (PLLA), but are not limited thereto. Preferably, the electrospun fibers may be poly(ε-caprolactone) (PCL), but are not limited thereto.

In addition, the fish oil may include omega-3 polyunsaturated fatty acids, but is not limited thereto.

The omega-3 polyunsaturated fatty acids may be at least one selected from the group consisting of Eicosapentaenoic acid (EPA), Docosahexaenoic acid (DHA), α-linolenic acid (ALA), docosapentaenoic acid (DPA), clupanodonic acid, tetracosapentaenoic acid, and tetracosahexaenoic acid, but is not limited thereto.

In one embodiment of the present disclosure, Menhaden fish oil from the genera Brevoortia and Ethmidium, two genera of the order Clupeiformes known to contain omega-3 polyunsaturated fatty acids, was used.

In the present disclosure, the leukotriene receptor antagonist may preferably be montelukast or a pharmaceutically acceptable salt thereof.

In the present disclosure, the term “pharmaceutically acceptable salt” means a salt in the form that may be used pharmaceutically among salts as materials in which cations and anions are bonded by electrostatic attraction, and may be generally a metal salt, a salt with an organic base, a salt with an inorganic acid, a salt with an organic acid, a salt with a basic or acidic amino acid, and the like. For example, the metal salt may be an alkali metal salt (sodium salt, potassium salt, etc.), an alkaline earth metal salt (calcium salt, magnesium salt, barium salt, etc.), an aluminum salt, etc. The salt with the organic base may be salts with triethylamine, pyridine, picoline, 2,6-lutidine, ethanolamine, triethanolamine, cyclohexylamine, diethanolamine, dicyclohexylamine, N, N-dibenzylethylenediamine, etc. The salt with the inorganic acid may be salts with hydrochloric acid, hydrobromic acid, nitric acid, sulfuric acid, phosphoric acid, etc. The salt with the organic acid may be salts with formic acid, acetic acid, trifluoroacetic acid, phthalic acid, fumaric acid, oxalic acid, tartaric acid, maleic acid, citric acid, succinic acid, methanesulfonic acid, benzenesulfonic acid, p-toluenesulfonic acid, etc. The salt with the basic amino acid may be salts with arginine, lysine, ornithine, etc., and the salt with the acidic amino acid may be salts with aspartic acid, glutamic acid, etc., but are not limited thereto. More preferably, the pharmaceutically acceptable salt of montelukast may be montelukast sodium.

The montelukast sodium is represented by the following Chemical Formula 1 and is effective in alleviating bronchial inflammation and preventing asthma symptoms.

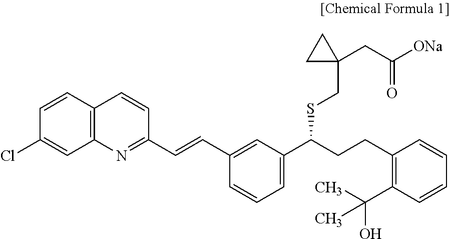

In the present disclosure, the fish oil and the leukotriene receptor antagonist may be encapsulated in the electrospun fibers.

The electrospun fibers encapsulating the fish oil and the leukotriene receptor antagonist of the present disclosure are characterized by exhibiting peaks at any one or more wavelengths selected from the group consisting of 1164 cm⁻¹, 1240 cm⁻¹, 1295 cm⁻¹, 1465 cm⁻¹, 1607 cm⁻¹, 1722 cm⁻¹, 2856 cm⁻¹, 2922 cm⁻¹, and 3012 cm⁻¹, when analyzed by Fourier-transform infrared spectroscopy (FT-IR).

This indicates a PCL-specific peak as well as fish oil and MTKS-specific peaks, suggesting that the electrospun fibers of the present disclosure contain fish oil and MTKS.

The electrospun fibers according to the present disclosure are characterized by having a smooth and thin morphology and an average diameter of 1 to 3 μm. Preferably, the electrospun fibers are characterized by having an average diameter of 2 μm.

In addition, the electrospun fibers according to the present disclosure are characterized by being hydrophilic. According to one embodiment of the present disclosure, it was confirmed that the electrospun fibers according to the present disclosure had a water contact angle of 40.81±1.63. Therefore, it is apparent that the electrospun fibers according to the present disclosure are a material on which bacterial adhesion and biofilm formation are difficult due to hydrophilicity.

In addition, the electrospun fibers according to the present disclosure are characterized by exhibiting a Braggs angle (2θ) of 17.5° when analyzed by X-ray diffraction analysis.

In addition, the electrospun fibers of the present disclosure are characterized by exhibiting thermal stability at 300 to 400° C.

According to an embodiment of the present disclosure, it was clearly confirmed that the electrospun fibers of the present disclosure had thermal stability even at high temperatures of 300 to 400° C., as one major degradation DTG peak was identified at 413° C. and two minor peaks were found at 469° C. and 480° C. Therefore, the electrospun fibers of the present disclosure are a material that is thermally very stable when inserted into the human body at 35 to 37° C.

In addition, the present disclosure provides a pharmaceutical composition for preventing or treating capsular contracture, including electrospun fibers containing fish oil and a leukotriene receptor antagonist.

The pharmaceutical composition of the present disclosure may further include a pharmaceutically acceptable carrier, an excipient or a diluent for administration, in addition to the fish oil and the leukotriene receptor antagonist. The carrier, the excipient, and the diluent may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil.

A preferable dosage of the pharmaceutical composition of the present disclosure varies according to the condition and body weight of a patient, the age, the degree of a disease, a drug form, and the route and period of administration, but may be properly selected by those skilled in the art. The dosage may be 0.0001 to 100 mg/kg per day, preferably 0.001 to 10 mg/kg, and the administration may be administered once a day or divided into several times, but is not limited thereto. The dosage of the pharmaceutical composition of the present disclosure, which is effective in preventing, improving, or treating capsular contracture, may be easily adjusted.

The composition of the present disclosure may be in any form for topical application, for example, a solution, a spray, or a transdermal device such as a storage patch type. Furthermore, the composition of the present disclosure may include any pharmaceutically acceptable excipient, such as a thickener, a fragrance, or a flavoring agent, or an alcohol compound, or an emollient to counteract the drying effect. Such an emollient may be selected from glycerol or propylene glycol, but are not limited thereto.

As used in the present disclosure, the term “administration” means introducing a predetermined substance into a patient in any suitable method, and an administration route of the compositions may be administered through any general route so long as the composition may reach a target tissue. In addition, the pharmaceutical composition of the present disclosure may also be administered by any device capable of transferring an active substance to a target tissue.

The pharmaceutical composition of the present disclosure may be administered as an individual therapeutic agent or administered in combination with other therapeutic agents, and sequentially or simultaneously administered with conventional therapeutic agents. The pharmaceutical composition may be administered in an amount capable of obtaining a maximum effect with a minimal amount without side effects by considering all the factors, which may be easily determined by those skilled in the art.

In addition, the present disclosure provides a quasi-drug composition for preventing or improving capsular contracture, including electrospun fibers containing fish oil and a leukotriene receptor antagonist.

In the present disclosure, the term “quasi-drug” means products that have a milder effect than drugs among products used for the purpose of diagnosing, treating, improving, alleviating, managing or preventing diseases of humans or animals. For example, according to the Pharmaceutical Affairs Act, the quasi-drug refers to a product excluding products used for the purpose of drugs, and includes fiber and rubber products used for the treatment or prevention of diseases of humans or animals, products that have a mild or no direct effect on the human body, are not instruments or machines or similar thereto, sterilizers and insecticides for preventing infectious diseases, etc.

The type or formulation of the quasi-drug composition of the present disclosure is not particularly limited, but may include a bandage, gauze, cotton wool, adhesive plaster, etc.

When the composition of the present disclosure is incorporated into an quasi-drug for the purpose of preventing or improving capsular contracture, the composition may be used as it is or in combination with other quasi-drug ingredients, and may be used appropriately according to conventional methods. The mixing amount of the active ingredients may be appropriately determined depending on the purpose of use.

Further, the present disclosure provides a composition for implantation including electrospun fibers containing fish oil and a leukotriene receptor antagonist.

The composition for implantation of the present disclosure is characterized by having an effect of preventing, improving or treating capsular contracture.

The composition for implantation of the present disclosure may be provided as a kit for preventing, improving or treating capsular contracture. The kit may include a description of the electrospun fibers of the present disclosure and its components, such as fish oil and a leukotriene receptor antagonist, and a description of the resulting effects of prevention, improvement, or treatment for capsular contracture, but is not limited thereto. In addition, the kit may further include a structure for implantation, preferably a biocompatible structure.

In addition, the present disclosure provides a method for preventing or treating capsular contracture in a subject other than a human, including implanting a composition for implantation, including electrospun fibers containing fish oil and a leukotriene receptor antagonist, into a subject other than a human.

In the present disclosure, the composition for implantation may further include a biocompatible structure. The biocompatible structure is able to be used with any substance known in the art to be stable in vivo, without limitation. For example, the biocompatible structure may be a structure consisting of at least one selected from the group consisting of gel-like materials, saline solution, silicone, and polyester, but is not limited thereto.

In the present disclosure, the leukotriene receptor antagonist and the fish oil are characterized to be released sequentially.

That is, the leukotriene receptor antagonist is first rapidly released to treat the early acute inflammation, and then the fish oil is continuously released to treat the inflammation, and the leukotriene receptor antagonist and the fish oil commonly cause the effect of preventing and treating capsular construction.

In addition, the electrospun fibers are characterized by inhibiting the activation of myofibroblasts and reducing collagen over-deposition or fibrosis over-production.

Through this, the electrospun fibers are characterized by developing the fibrosis of the surrounding implanted tissue to a late stage. That is, when the electrospun fibers of the present disclosure are implanted, the collagen fiber of the surrounding tissue rapidly progresses to a late stage of fibrosis so as to exhibit an isotropic structure. More specifically, when the electrospun fibers of the present disclosure are implanted, the collagen fiber of the surrounding implanted tissue is characterized by changing from an anisotropic structure to an isotropic structure.

This is because the fish oil and the leukotriene receptor antagonist contained in the electrospun fibers of the present disclosure rapidly progress to the early stage of fibrosis and further rapidly progress to the late stage of fibrosis, thereby causing an effect of reducing the thickness of the capsule.

In addition, the electrospun fibers are characterized by inhibiting bacterial adhesion and biofilm formation.

In addition, the electrospun fibers are characterized by reducing the expression of any one or more selected from the group consisting of Connective Tissue Growth Factor (CTGF), interferon gamma (INF-γ), Matrix Metalloproteinase-9 (MMP-9), Cluster of Differentiation 68 (CD68), and tumor necrosis factor-α (TNF-α), which are increased by capsular contracture. In addition, the electrospun fibers are characterized by increasing the expression of any one or more selected from the group consisting of galectin-1, collagen type I alpha 1 chain (COL1A1), and transforming growth factor-beta 1 (TGF-β1), which are decreased by capsular contracture.

That is, the electrospun fibers effectively suppress an increase in inflammatory cytokines caused by the formation of capsular contracture, and exhibit balanced collagen regulation to stabilize the tissue structure by balancing tissue repair and fibrosis.

Disordered or excessive collagen deposition causes the formation of capsular contracture, but a balanced collagen matrix prevents the formation of capsular contracture and supports structural integrity. Therefore, the electrospun fibers of the present disclosure are characterized by effectively preventing, improving, and treating various conditions of capsular contracture diseases.

Further, the present disclosure provides a prosthesis including electrospun fibers containing fish oil and a leukotriene receptor antagonist; and a biocompatible structure.

The biocompatible structure is able to be used with any substance known in the art to be stable in vivo, without limitation. For example, the biocompatible structure may be a structure consisting of at least one selected from the group consisting of gel-like materials, saline solution, silicone, and polyester, but is not limited thereto.

Furthermore, the present disclosure provides a method for preparing electrospun fibers containing fish oil and a leukotriene receptor antagonist, including dissolving electrospun fibers in a solvent; preparing a first mixture by mixing fish oil into the solvent in which the electrospun fibers are dissolved; preparing a second mixture by adding a leukotriene receptor antagonist to the first mixture; and spinning the second mixture using an electrospinner.

In the present disclosure, the electrospun fibers may be at least one selected from the group consisting of poly(ε-caprolactone) (PCL), polyvinyl alcohol (PVA), polystyrene (PS), poly(lactic-co-glycolic) acid (PLGA), and poly(l-lactic acid) (PLLA), but is not limited thereto. Preferably, the electrospun fibers may be poly(ε-caprolactone) (PCL), but are not limited thereto.

In addition, the fish oil may include omega-3 polyunsaturated fatty acids, but is not limited thereto.

The omega-3 polyunsaturated fatty acids may be at least one selected from the group consisting of Eicosapentaenoic acid (EPA), Docosahexaenoic acid (DHA), α-linolenic acid (ALA), docosapentaenoic acid (DPA), clupanodonic acid, tetracosapentaenoic acid, and tetracosahexaenoic acid, but are not limited thereto.

In one embodiment of the present disclosure, Menhaden fish oil from the genera Brevoortia and Ethmidium, two genera of the order Clupeiformes known to contain omega-3 polyunsaturated fatty acids, was used.

In the present disclosure, the leukotriene receptor antagonist may preferably be montelukast or a pharmaceutically acceptable salt thereof. More preferably, the pharmaceutically acceptable salt of montelukast may be montelukast sodium.

In the present disclosure, the solvent is characterized as a mixed solvent of chloroform and methanol, and the chloroform and methanol may be preferably mixed in % v/v of 1 to 2:1, and more preferably, mixed in % v/v of 1.5:1.

In electrospinning, the solvent is a factor that affects the properties of electrospun fibers. The morphology, diameter, beads, etc. of the fibers may vary according to the type, concentration, volatility, etc. of the solvent.

In the method for preparing the electrospun fibers of the present disclosure, the electrospun fibers may be included in an amount of 11 to 13% (w/v), but are not limited thereto. Preferably, the electrospun fibers may be included in an amount of 12% (w/v), but are not limited thereto.

In addition, the fish oil may be included in an amount of 25 to 55% (w/v), but is not limited thereto. According to one embodiment of the present disclosure, when 60% (w/v) of fish oil was mixed with 12% (w/v) of fibers, it was confirmed that an organic phase having two immiscible layers was generated. Accordingly, the electrospun fibers of the present disclosure are characterized by including fish oil at a concentration of up to 55% (w/v), preferably 50% (w/v).

In addition, the leukotriene receptor antagonist may be included in an amount of 0.5 to 4% (w/v), but is not limited thereto. The leukotriene receptor antagonist may be included in an amount of preferably 1 to 3% (w/v), and more preferably 2% (w/v), but is not limited thereto.

In addition, the electrospun fibers are characterized by being spun under conditions of a feed rate of 2 to 5 ml/hr and an applied voltage of 10 to 30 kV. Preferably, the electrospun fibers may be spun under conditions of a feed rate of 3.5 to 4.5 ml/hr and an applied voltage of 15 to 25 kV, and more preferably, a feed rate of 4 ml/hr and an applied voltage of 20 kV, but is not limited thereto.

The electrospun fibers of the present disclosure may be prepared by electrospinning in an environment of 30 to 60% humidity, preferably 45 to 55% humidity, and more preferably 50% humidity, but is not limited thereto.

Furthermore, the present disclosure provides a method for preparing a breast implant composition including: dissolving electrospun fibers in a solvent; preparing a first mixture by mixing fish oil into the solvent in which the electrospun fibers are dissolved; preparing a second mixture by adding a leukotriene receptor antagonist to the first mixture; spinning the second mixture using an electrospinner; and coating the surface of a biocompatible structure with the spun electrospun fibers.

The biocompatible structure is able to be used with any substance known in the art to be stable in vivo, without limitation. For example, the biocompatible structure may be a structure consisting of at least one selected from the group consisting of gel-like materials, saline solution, silicone, and polyester, but is not limited thereto.

Throughout this specification, ‘%’ used to indicate the concentration of a specific material is solid/solid (w/w) %, solid/liquid (w/v) %, and liquid/liquid (v/v) %, unless otherwise stated.

Terminologies used in the present disclosure are terminologies used to properly express preferred embodiments of the present disclosure, which may vary according to a user, an operator's intention, or customs in the art to which the present disclosure pertains. Accordingly, definitions of the terminologies need to be described based on contents throughout this specification. Throughout the specification, when a part “comprises” a certain component, it is meant that the part may further include other components, not excluding other components, unless explicitly described to the contrary.

All technical terms used in the present disclosure, unless otherwise defined, are used as the meaning as commonly understood by those skilled in the related art of the present disclosure. In addition, although preferred methods and samples are described in the present disclosure, similar or equivalent methods and samples thereto are also included in the scope of the present disclosure. The contents of all publications disclosed in the present disclosure by reference are incorporated in the present disclosure.

Hereinafter, embodiments of the present disclosure will be described in detail with reference to the accompanying drawings. However, the following exemplary embodiments are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure may be made within the description of claims to be described below and the equivalent scope interpreted therefrom.

### Preparation Example 1. Experimental Materials

PCL pellets (average Mn 80,000) and fish oil were purchased from Menhaden, and MTKS was purchased from Sigma-Aldrich, Korea. Chloroform (CHCl₃), methanol (MeOH), and ethyl acetate (EA) were purchased from SK Chemicals, Korea. Deionized (DI) water was used in the experiments.

### Example 1. Preparation of the Electrospun Fibers

PCL 12% (w/v) was added to a mixture of CHCl₃:MeOH (3:2, v/v), and the solution was stirred using a vortex mixer until the polymer was fully dissolved. Fish oil (50% w/v) was added to the PCL solution to prepare a ω3-containing electrospinning solution (PCL-ω3 fiber). PCL-ω3-MTKS fiber was prepared by adding 2% (w/v) MTKS to the PCL-ω3 fiber. The prepared solution was immediately injected in a 5 ml Luer-slip syringe fitted with a 22-gauge plastic nozzle with a 0.4 mm inner diameter (NanoNC, Seoul, Korea). The syringe was mounted on a syringe pump and clamped to a high-voltage power supply using alligator clips in an electrospinner (ESR200R2D, NanoNC, Seoul, Korea). The feed rate and the applied voltage were held constant at 4 ml/hr and 20 kV, respectively. The distance between the needle tip and the collector plate was adjusted to 13 cm. To collect the as-spun fibers, a clean aluminum foil (30 cm×30 cm) was placed on the metal collector plate. All samples were electrospun for 1 hour at room temperature in a 50% humid environment.

### Experimental Example 1. Analysis Method

### 1-1. Fourier-Transform Infrared Spectroscopy (FT-IR)

The structure and composition of the electrospun fibers were characterized by FT-IR analysis. The analysis was performed using a JASCO FT/IR-4600 spectrometer equipped with an attenuated total reflectance (ATR) technique. The as-spun fibers, and solid PCL, MTKS, and liquid fish oil, were analyzed using FT-IR spectroscopy. The samples (about 0.1 mg) were spread on the ATR crystal and scanned 32 times in a frequency range of 4000 to 500 cm⁻¹ with constant pressure using a pressure device for an even spread. Each sample was analyzed three times at room temperature.

### 1-2. Scanning Electron Microscopy (SEM)

The morphology of the fibers was investigated by SEM (Axia ChemiSEM LoVac, Thermo Fischer Scientific, Waltham, MA, USA) study. Fiber samples were collected in a small piece of silicon wafer and sputter-coated with gold before SEM analysis. The fiber diameter was measured using ImageJ software (NIH, Bethesda, MD, USA) and plotted as a histogram using Origin Pro 2018 software (OriginLab Corp., Northampton, MA, USA).

### 1-3. Measurement of Water Contact Angle (WCA)

The surface wettability of the fibers was monitored by contact angle measurement study (DSA, KRUSS GMBH, Hamburg, Germany). First, DI (about 3 μL) was dropped onto the fiber sample using a micro-syringe and a picture of the water droplet was taken. The average WCA value was determined by taking three measurements at different locations on the same fiber surface.

### 1-4. X-Ray Diffraction (XRD) Analysis

The crystallinity of PCL in the prepared fibers was determined using XRD (Rigaku MiniFlex-600, Rigaku corp., Tokyo, Japan). Samples were spread over the holder and scanned at a rate of 10°/minute in a 2θ range of 5° to 45°. The XRD peak of solid PCL was first determined and then compared to the XRD peak of the fiber samples to monitor a change in crystallinity of PCL during the fiber preparing process. PCL peaks in the spectrum were also identified and the XRD peaks of MTKS were also assessed and compared.

### 1-5. Thermogravimetric Analysis (TGA)

The thermal behavior of fibers and fiber precursors (PCL, fish oil, and MTKS) were analyzed using TGA analysis (TA Q50 thermal analyzer, TA instruments, New Castle, DE, USA). The analysis was performed in a temperature range of 30° C. to 800° C. at a constant heating rate of 10° C./minute under an N₂ atmosphere. Before analysis, 4 mg of the sample was placed on a clean platinum pan. The onset temperature (Tᵢ) was determined by locating a point where the sample weight loss was high and steep. The derivatives (DTG; %/° C.) of TGA curve were made by differentiating the values obtained from each TGA spectrum in Origin Pro 2018 software.

1-6. Selective Removal of Fish Oil in as-Spun Fiber

A selective removal was performed to identify the location of fish oil in the as-spun fiber. First, the fish oil was selectively removed from the fiber, and then the resulting fibers were analyzed. Hexane was used to wash the fibers in order to selectively remove fish oil while keeping the PCL comparatively intact. A small amount of the fiber sample containing fish oil was washed with hexane and dried before FT-IR analysis. The FT-IR peaks of the hexane-washed samples were compared with those of an unwashed fiber to confirm changes in the characteristic peaks. In addition, after the FT-IR analysis, the fiber morphology was examined using SEM. As a control, similar experiments were performed using a PCL fiber sample without fish oil.

### 1-7. Drug Encapsulation Efficiency

The drug encapsulation efficiency (EE) was investigated to inspect the amount of fish oil or MTKS inside the as-spun fiber. 10 mg of the dried electrospun fibers was taken in a 40 ml vial and dissolved completely in 10 ml of a binary solvent (CHCl₃:MeOH). The amount of released fish oil/MTKS was determined using a JASCO V-730 UV-Vis spectrometer at 263 nm for fish oil or at 284 nm for MTKS, which was the Amax of each compound. EE (%) was calculated using the following Equation 1.

$$EE ( \% ) = \frac{Amount of drug released from 10 mg fiber}{Theoretical amount of drug loaded in 10 mg fiber} \times 100 \qquad [ Equation 1 ]$$

### 1-8. In-Vitro Drug Release Study and Release Kinetic Modeling

In vitro drug release studies of the fibers were performed using a membrane dialysis method. The release of fish oil was monitored from both PCL-ω3 and PCL-ω3-MTKS fibers, whereas the release of MTKS was monitored using the PCL-ω3-MTKS fiber. In brief, a small piece of the as-spun fiber was inserted in a presoaked dialysis membrane (MWCO 1 kD), and the tube was sealed on both ends with dialysis closure. Then, the setup was immersed into a 500 ml beaker containing DI (pH 7) as a primary release medium. DI was used to mimic an aqueous environment around the human breast tissues. Since the fish oil is not miscible with water, EA was placed on the water layer to extract the released fish oil in DI.

The temperature was set to 37° C. to mimic physiological conditions, and the release medium was constantly stirred at 60 rpm. To investigate the release of MTKS, only DI was used as a release medium. At an hourly interval, 0.5 ml of the release medium was collected to assess the amount of released drug using UV-Vis spectroscopy. The aliquot was returned after each experiment to maintain a constant volume throughout the experiment. As there was no change in the volume of the aliquots in the short period during UV-Vis analysis, there was no need to further add a fresh release medium. For a better understanding of the release behavior of drugs (fish oil and MTKS) encapsulated inside the PCL fiber, the drug release data of fish oil and MTKS were applied to various kinetic models. Five different kinetic models (zero order, first order, Hixson Crowell, Korsmeyer-Peppas, and Higuchi models) were used in this study to understand the release kinetics. The R² values calculated in various models were compared, and the model that showed an R² value closest to 1 was considered the best fit.

### 1-9. Design of In Vivo Animal Study

Seven-week-old female Wistar rats (N=40; mean weight=195.7±8.0 g across all groups) were obtained from specific-pathogen-free colonies (DBL Co., Ltd, Eumseong, Korea) and were housed under standard laboratory conditions at a temperature of 22±2° C. with a relative humidity of 50±15% and a 12-hour light/dark cycle. The rats were freely fed with 2018C rodent feed (Teklad Diet, USA) and filtered, UV sterilized drinking water. The number of animals required in each group was determined using G*Power software (version 3.1). A priori power analysis was conducted using a F-test for ANOVA (fixed effects, omnibus, one-way). The parameters were set as follows: effect size (f)=0.55, significance level (α)=0.05, power (1−β)=0.80, and 4 experiment groups. The analysis results were a noncentrality parameter (λ)=13.31, a critical F7 value=2.84, numerator degrees of freedom=3, and denominator degrees of freedom=40. Based on this calculation, 10 rats were allocated to each group (G1, G2, G3, and G4), resulting in a total of 40 rats. The control group (G1) received uncoated silicone mini-implants, while another control group (G2) received mini-implants coated with neat PCL fiber. G3 received silicone mini-implants coated with PCL-ω3 fiber, and G4 received silicone mini-implants coated with PCL-ω3-MTKS fiber. At this time, in the mini-implants, the as-spun fibers were cut into small pieces (5×5 cm) and wrapped around spherical silicone mini-implants. The surgery was conducted under general anesthesia using isoflurane (induction: 48, maintenance: 1.5 to 2%). A 2 cm incision was made in the dorsal lumbosacral region, and the implants were placed subcutaneously under the panniculus carnosus layer, and the incisions were sutured using 4-0 black silk (Ethicon, NJ, USA). After the surgery, rats were closely monitored for behavior, appetite, and general health conditions, and maintained at recovery and normal activity levels. In 90 days of post-surgery, the rats were euthanized using carbon dioxide inhalation and then subjected to cervical dislocation. Capsule tissues surrounding the implants were carefully harvested, washed in PBS, and either fixed in 10% formaldehyde for histological and immunohistochemical analyses or frozen-stored for RNA extraction and qRT PCR studies of fibrosis and immune-related markers. Animal care and experimental procedures were authorized and conducted in accordance with the ethics guidelines and approval of the Institutional Animal Care and Use Committee of the WOOJUNG BIO (Approval No. IACUC2403-014; date of approval: Jul. 17, 2024).

### 1-10. Histological Analysis

The capsular contracture tissue surrounding the implants was excised, washed with phosphate-buffered saline (PBS), and fixed in 10% neutral buffered formalin. All capsular contracture tissues from each animal were analyzed, and the thickness of the capsular contracture tissue was consistently measured at the central region to standardize data collection. Histological analysis was performed on 4 μm-thick paraffin-embedded sections using hematoxylin and eosin (H&E), Masson's trichrome (MT), and immunohistochemistry (IHC). α-Smooth muscle actin was detected using primary antibodies (1:100 dilution, ab5694; Abcam, Cambridge, UK), and used with diaminobenzidine (DAB) development and hematoxylin counterstaining.

1-11. Quantitative Reverse Transcription Polymerase Chain Reaction (qRT-PCR)

Gene expression analysis of profibrotic and inflammatory genes, including galectin-1 (Lgals1), connective tissue growth factor (CTGF), transforming growth factor-beta 1 (TGF-31), collagen type I alpha 1 chain (COL1A1), matrix metalloproteinase 9 (MMP9), interferon-gamma (IFN-γ), tumor necrosis factor-alpha (TNF-α), and cluster of differentiation 68 (CD68), was performed using qRT-PCR. Tissues were frozen, homogenized in a TRIzol reagent (Thermo Fisher Scientific, Waltham, MA, USA), and processed for RNA extraction according to the following the manufacturer's protocol. The extracted RNA was converted into kit complementary DNA (CDNA) using a reverse transcription (Applied Biosystems, Foster City, CA, USA) and analyzed using the CFX Duet Real-Time PCR Detection System (Bio-Rad, Hercules, CA, USA). The housekeeping gene β-actin was used for normalizing the the qRT-PCR analysis. Consistent gene expression data in expression of β-actin in all samples ensured reliability of normalization and accurate quantification of target genes. The primers used for the qRT-PCR analysis were listed in Table 1 below. All samples were tested in duplicate, and relative gene expression was analyzed using cycle threshold (Ct) values and a Livak method (2^{-ΔΔCt}).

TABLE 1

| | Forward Sequence | Reverse Sequence |
|---|---|---|
| Target gene | (5′-3′) | (5′-3′) |
| β-actin | CATCATGAAGTGTGACGTTGAC | GCATCCTGTCAGCAATGCC |
| *Housekeeping | (SEQ ID NO. 1) | (SEQ ID NO. 2) |
| gene | | |
| Lgals1 | TGCAACAGCAAGGACGGC | CACCTCTGCAACACTTCCA |
| | (SEQ ID NO. 3) | (SEQ ID NO. 4) |
| CTGF | ATAGTTCACCACGGAAACACCTG | GCAGCAACCTTCTCCTTTAATG |
| | (SEQ ID NO. 5) | (SEQ ID NO. 6) |
| TGF-β1 | CCCAGCATCTGCAAAGCTC | GTCAATGTACAGCTGCCGCA |
| | (SEQ ID NO. 7) | (SEQ ID NO. 8) |
| COL1A1 | GAGCGGAGAGTACTGGATCG | GCTTCTTTTCCTTGGGGTTC |
| | (SEQ ID NO. 9) | (SEQ ID NO. 10) |
| MMP9 | AGGTGCCTCGGATGGTTATCG | TGCTTGCCCAGGAAGACGAA |
| | (SEQ ID NO. 11) | (SEQ ID NO. 12) |
| IFN-γ | CGAAAAGCTGACTAATTATTCGG | CTCTTCGACCTCGAAACAGC |
| | (SEQ ID NO. 13) | (SEQ ID NO. 14) |
| TNF-α | AAGCCTGTAGCCCACGTCGTA | GGCACCACTAGTTGGTTGTCTTTG |
| | (SEQ ID NO. 15) | (SEQ ID NO. 16) |
| CD68 | TGTTGCGGAAATACAAGCA | GGCAGCAAGAGAGATTGGTC |
| | (SEQ ID NO. 17) | (SEQ ID NO. 18) |

### 1-12. Second Harmonic Generation (SHG) Imaging Study

The distribution of fibrillar collagen within capsular contracture tissues was confirmed in various experimental groups of the present disclosure using SHG imaging microscopy. A femtosecond laser (Carmel X-780, Calmar Laser) with a wavelength of 780 nm was guided by a high-speed two-axis Galvano mirror (GVS002, Thorlabs). The beam was expanded using a combination of plano-convex lenses in a telecentric configuration and then focused onto a sample through a 40× objective lens (CFI Plan Apo Lambda, Nikon). Nonlinear optical signals were filtered by a bandpass filter (FF01-392/23, Semrock) centered at 390 nm and detected with a photon-counting head photomultiplier tube (H10682-210, Hamamatsu). SHG images of collagen were acquired at a resolution of 512×512 pixels over an area of 240×240 μm². For analysis, tissue specimens were excised from the peri-implant region, immersed in PBS, and fixed in imaging Petri dishes. SHG images obtained from each experimental group were analyzed using ImageJ software with Fiji OrientationJ plugin. The orientation angles of fibrillar collagen and the degree of correlation across all SHG images were collected. To determine a fiber angle, the images were first converted to 8-bit images and then processed using OrientationJ Distribution plugin tool. The structure tensor was set as Gaussian window σ 1 pix and Gaussian gradient.

### 1-13. Statistical Analysis

All values were expressed as mean±standard deviation. Statistical analysis of the experimental data was performed using StatView software (version 4.51; Abacus Concepts, Berkeley, CA, USA). Comparisons between experimental groups were conducted using analysis of variance (ANOVA), and Fisher's protected least significant difference (PLSD) applied when statistical significance was observed. Statistical significance was evaluated at levels of p<0.05, p<0.01, and p<0.001, and these levels were indicated on the respective graphs. qRT-PCR results were normalized to a G1 (implant only) experimental group and presented as fold-change values.

### Example 2. Characterization of Electrospun Fibers

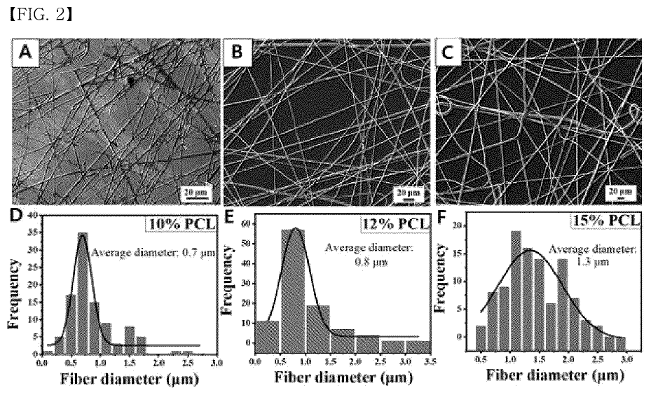

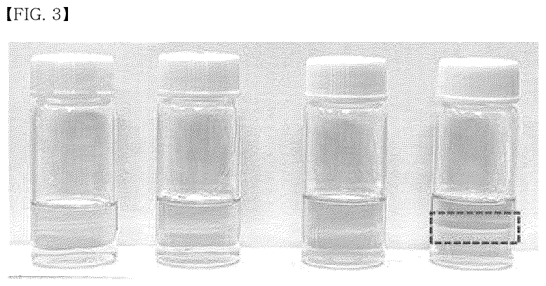

In the present disclosure, PCL was used as the main fiber matrix to prepare electrospun fibers encapsulating fish oil and a leukotriene receptor antagonist MTKS. Since the fiber morphology may be tuned by altering the amount of polymer, the suitable amount (% w/v) of PCL required for stable fiber formation was investigated. Menhaden fish oil was an abundant source of ω3 PUFA, such as EPA and DHA, and used as anti-inflammatory agents in the present disclosure. Solutions were prepared and electrospun by adding fixed 1 g of fish oil into various concentrations (10%, 12%, 15% w/v) of 5 ml PCL, and as a result, a 12% (w/v) PCL solution provided the most stable jet and consistent fiber diameters (FIG. 2). It is very important to maintain a uniform morphology of fibers and enhance the encapsulation efficiency. Then, various amounts of fish oil (30%, 40%, 50%, and 60% w/v, (each, 1.5 g, 2 g, 2.5 g, 3 g)) were mixed with a 12% (w/v) PCL solution to derive the conditions for maximally incorporating fish oil into the electrospun fibers. It was confirmed that when 60% w/v of fish oil was mixed, an organic phase with two immiscible layers was formed. Accordingly, it was found that a maximum of 50% (w/v) fish oil could be added into the 12% (w/v) PCL solution (FIG. 3). Therefore, the optimal formulation of the present disclosure was determined to contain 12% (w/v) PCL and 50% (w/v) fish oil. 2% (w/V) MTKS was also added to the solution before electrospinning to fabricate PCL-ω3-MTKS fiber. After electrospinning, the as-spun fibers and their fibrillar mats appeared consistently in smooth and lightweight forms without defects or beads.

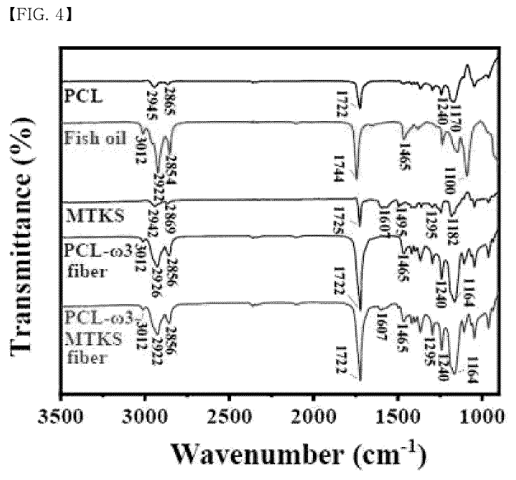

The components and bonding characteristics of the as-spun PCL-ω3 and PCL-ω3-MTKS fibers were verified using FT-IR analysis. The FT-IR spectra of each fiber precursor (PCL, fish oil, MTKS) and both of the as-spun fibers were shown in FIG. 4. PCL shows characteristic FT-IR peaks at 2945 cm⁻¹ and 2865 cm⁻¹ due to asymmetric and symmetric vibration of C—H bonds. The peak at 1724 cm⁻¹ represents carbonyl C═O vibration, and peaks at 1240 cm⁻¹ and 1167 cm⁻¹ represent stretching of C—O—C bonds. The characteristic FT-IR peak of fish oil is observed at 3012 cm⁻¹ due to stretching vibration of ═C—H bond from a cis olefin group. The peaks at 2922 cm⁻¹ and 2854 cm⁻¹ in fish oil are caused due to the asymmetric and symmetric stretching vibration of CH₂ groups, similar to PCL. The peak at 1744 cm⁻¹ represents the C═O stretching vibration, while 1463 cm⁻¹ represents the bending vibration of the C—H bond. In addition, the sharp peak at 1100 cm⁻¹ indicates the presence of-C—O vibration in fish oil. The peak at 871 cm⁻¹ represents C—H stretching. Another distinct peak at 721 cm⁻¹ appears due to the rocking vibration of a methylene (—CH₂) bond. As shown in FIG. 4, PCL-ω3 and PCL-ω3-MTKS fibers showed all the characteristic peaks of the neat PCL and fish oil. Both of the fibers showed FT-IR peaks at 1240 cm⁻¹ and 1164 cm⁻¹ due to the presence of C—O—C bonds, which matched well with the peak of PCL. Also, the fibers showed peaks at 3012 cm⁻¹, 2926 cm⁻¹, and 2856 cm⁻¹ due to the presence of cis olefinic=C—H bond and symmetric/asymmetric C—H bonds. These peaks matched well with the peaks of fish oil to indicate that fish oil was present in all as-spun fibers. The neat solid MTKS showed FT-IR peaks at 2942 cm⁻¹ and 2869 cm⁻¹ due to the presence of asymmetric and symmetric stretching vibration of C—H groups. The peak around 1725 cm⁻¹ arises from the C═O stretching. The peaks at 1607 cm⁻¹, 1495 cm⁻¹, 1295 cm⁻¹, and 1182 cm⁻¹ indicate the presence of C═C stretching, C—H (aromatic), C—N stretching and C—O stretching, respectively. As shown in FIG. 4, PCL-ω3-MTKS fiber shows major peaks at 1722 cm⁻¹, 1607 cm⁻¹, 1295 cm⁻¹, and 1295 cm⁻¹, confirming the presence of MTKS. Collectively, the FT-IR spectra demonstrated the successful encapsulation of fish oil and MTKS within the PCL matrix, which was indicated by characteristic peaks in the as-spun fiber.

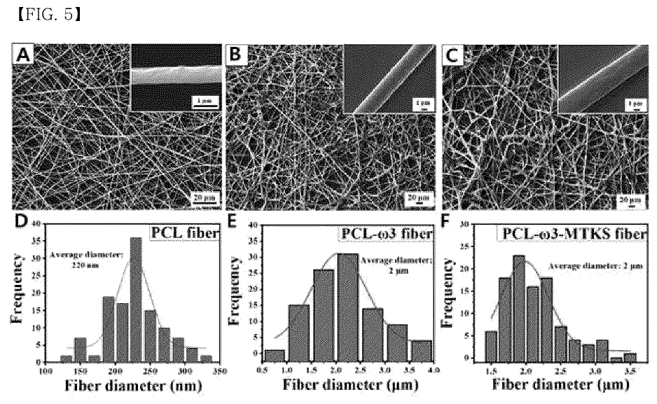

The morphology of PCL-ω3 and PCL-ω3-MTKS fibers was inspected by SEM analysis. As shown in (A) to (F) of FIG. 5, the as-spun fibers appeared smooth and thin in texture. The fiber diameter of neat PCL was found to be 220 nm and remarkably increased after adding fish oil and MTKS. The average diameter was measured to be 2 μm for both PCL-ω3 and PCL-ω3-MTKS fibers. Although the diameter of the as-spun fibers increased significantly, drug aggregates or humps were not visually identified on the surface of the fibers. Therefore, it was confirmed that all fish oil and MTKS were well incorporated inside the as-spun fibers prepared by the optimized electrospinning conditions.

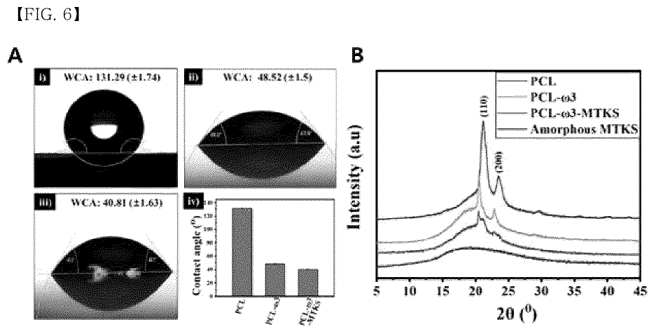

The first stage of CC often involves formation of a bacterial biofilm around the implant, which eventually triggered the inflammatory responses, and the immune system in the body prepared to make a thick fibrillar tissue to cope with the situation. The formation of the bacterial biofilm after bacterial adhesion to the silicon implant is related to a series of surface parameters, such as surface charge density, surface wettability, surface roughness, etc. Recently, it was found out that bacterial growth was reduced in the hydrophilic surface, compared to the hydrophobic surface. The WCA study was performed to examine the wettability of the as-spun PCL-ω3 and PCL-ω3-MTKS fibers since the mini-implant of the present disclosure had a structure in which the surrounding area of the silicone implant was coated with the fibers. In addition, the WCA of neat PCL fiber was also compared to observe a change in wettability after the drugs were incorporated. As shown in (A) (i) of FIG. 6, the contact angle of PCL fiber was found to be 131.29±1.74, which indicated the extreme hydrophobility of PCL. In comparison, the WCA of PCL-ω3 and PCL-ω3-MTKS fibers was measured to be 48.52±1.5 and 40.81±1.63 ((A) (ii) and (A) (iii) of FIG. 6), which indicated that the surface was relatively hydrophilic. Accordingly, when fish oil and MTKS were incorporated in the PCL fiber, the fibrillar surface became hydrophilic ((A) (i) to (A) (iv) of FIG. 6) to form a hydration layer. The possibility of bacterial adhesion and protein adsorption was reduced by the hydration layer, which reduced bacterial growth and biofilm formation, and also increased the bioavailability of the fibers.

In addition, the crystallinity of the fibers was characterized through XRD study. As shown in (B) of FIG. 6, PCL fiber showed two sharp peaks at Braggs angles (2θ) of 21.3° and 23.5° due to the diffractions at (110) and (200) crystalline planes. MTKS showed a hump in the range of 10 to 30°, showing its amorphous nature. In both as-spun fibers PCL-ω3 and PCL-ω3-MTKS, both peaks of PCL were present. This result indicated that the presence of fish oil did not affect largely the crystallinity of PCL. The broad peak around 17.5° observed in PCL-ω3 and PCL-ω3-MTKS fibers indicated the incorporation of fish oil and MTKS.

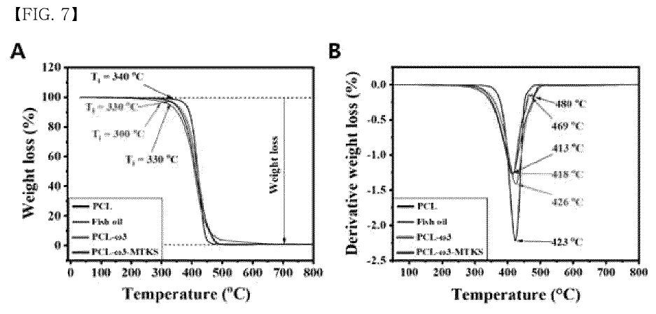

Thermal stability of the electrospun fibers is crucial for their biological applications. The fibers are required to be thermally stable at 35 to 37° C. when inserted into the human body for treatment. The thermal reactivity of each as-spun fiber was analyzed and compared with the thermal reactivity of the precursors using TGA. As a result, as shown in (A) of FIG. 7, the overlaid TGA spectra of PCL, fish oil, and the as-spun fibers PCL-ω3 and PCL-ω3-MTKS were shown. The onset temperature (Ti) for each sample was determined to indicate the temperature at which major transitions begin to occur. Ti for PCL was found to be 320° C., whereas Ti for fish oil to be 280° C. Both PCL-ω3 and PCL-ω3-MTKS indicated a Ti value of 290° C. as the major degradation step, much higher than the human body temperature. For a better interpretation of the thermal spectra, the DTG graph was also plotted ((B) of FIG. 7). PCL showed a single DTG peak at 423° C., indicating the maximum degradation of the polymer. For fish oil, the DTG peak was found at 426° C. PCL-ω3 fiber showed one sharp peak at 418° C. For PCL-ω3-MTKS fiber, one major degradation DTG peak was identified at 413° C., and two small peaks were found at 469° C. and 480° C. These results collectively indicated that the as-spun fibers exhibited excellent thermal properties, remained stable below 300 to 400° C., which was ideal for applications requiring thermal stability.

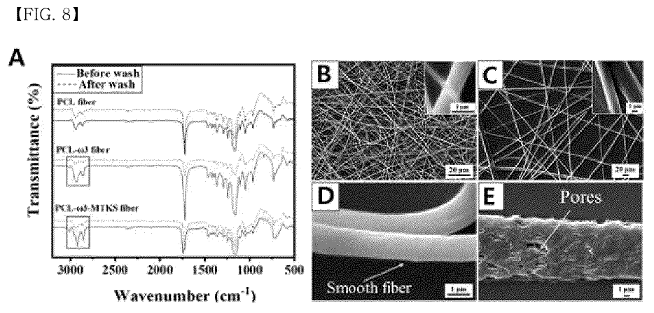

Through FT-IR study, all as-spun fibers were analyzed for the presence of fish oil after washing with hexane. As shown in (A) of FIG. 8, since the PCL fiber originally did not contain fish oil and was not affected by the selective removal, the FT-IR peaks of the PCL fiber were not changed. However, the characteristic FT-IR peak of fish oil at 3012 cm⁻¹ disappeared, as a result of washing the fish oil-containing fibers with hexane other than the PCL fiber.

Furthermore, the morphology change of the as-spun fibers after washing was investigated by SEM. As shown in (B) and (C) of FIG. 8, both PCL and PCL-ω3 fibers showed smooth surface before washing. However, after washing the fibers with hexane, the PCL-ω3 fiber exhibited pores in the surface while the PCL fiber was not damaged ((D) and (E) of FIG. 8). The appearance of pores in the PCL-ω3 fiber surface meant that the fish oil was well absorbed in the PCL-ω3 fiber and was washed with hexane to be selectively removed. However, the PCL fiber was not affected due to no fish oil. A nonpolar solvent, hexane may be assumed to mimic the hydrophobic surface of a cell membrane, and accordingly, it may be expected that the fish oil may be easily eluted from the as-spun fiber in the human body. As a result, it was confirmed that the fish oil was encapsulated in the PCL-ω3 fiber.

### Example 3. Encapsulation Efficiency (EE), In Vitro Drug Release Study, and Release Kinetics Modeling

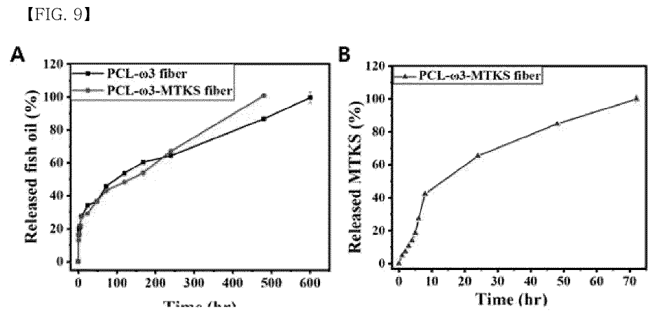

EE is another important factor to consider while preparing a drug-loaded formulation. The EE determined the effective amount of drug that was loaded in the final formulation and provided the idea of the cost-effectiveness that the formulation may provide in delivering the active drug. As shown in FIG. 9, the EE of fish oil and MTKS was found to be >98% in the as-spun fibers PCL-ω3 and PCL-ω3-MTKS. This indicates that the active drugs were well encapsulated inside the PCL fiber, making the formulation preferable.

As fish oil and MTKS were used as drugs in the fibers of the present disclosure, the in vitro drug release behavior of the drugs was investigated using a membrane dialysis method using a dialysis membrane in water media. The released fish oil from the as-spun fiber was extracted to EA, and the amount of released drug was calculated based on UV-Vis spectroscopy. For MTKS, a water layer was collected and examined since MTKS was well-soluble in water. As shown in (A) of FIG. 9, fish oil was released from both PCL-ω3 and PCL-ω3-MTKS fibers in a controlled and sustainable manner. The fish oil was completed released after 450 hours (18 days) in the case of PCL-ω3 fiber, and 600 hours (25 days) in the case of PCL-ω3-MTKS fiber. In the case of MTKS, the complete release from PCL-ω3-MTKS fiber occurred rather fast with 100% release by 72 hours (3 days) ((B) of FIG. 9).

Through this, it was confirmed that the as-spun fibers of the present disclosure rapidly released MTKS to treat early acute inflammation and then continuously released fish oil to exhibit long-term anti-inflammatory effects to help prevent CC.

The drug release data of fish oil and MTKS were further confirmed using various kinetic models to get more insights into the drug release behavior. The parameters obtained from various kinetic models were shown in Table 2 below. The R² value for each model was compared, and a model with a value closest to 1 was determined as the best-fitted model for the corresponding drug release. The R² value for the best-fitted kinetic model for each experiment was underlined. As shown in Table 2 below, 1 and 2 represent the fish oil release from both PCL-ω3 and PCL-ω3-MTKS fibers, respectively, and 3 represents the MTKS release from the PCL-ω3-MTKS fiber.

TABLE 2

| | Zero-order | | | First order | | | Hix [symbol] on Crowell | | Korsmeyer-Pepp [symbol] s | | | Higuch [symbol] | |
|---|---|---|---|---|---|---|---|---|---|---|---|---|---|
| No. [symbol] | K [symbol] | M [symbol] | R [symbol] | K [symbol] | M [symbol] | R [symbol] | K [symbol] | R [symbol] | n | K [symbol] | R [symbol] | K [symbol] | R [symbol] |
| 1 | 0.133 | 26.68 | 0.928 | 0.008 | 3.601 | 0.830 | 0.0127 | 0.972 | 0.264 | 1.447 | 0.984 | 3.422 | 0.966 |
| 2 | 0.225 | 18.35 | 0.926 | 0.011 | 3.387 | 0.904 | 0.0068 | 0.554 | 0.298 | 1.355 | 0.961 | 2.571 | 0.947 |
| 3 | 0.277 | 2.776 | 0.903 | 0.0 [symbol] 0 | 2.957 | 0.993 | 0.0223 | 0.754 | 0.745 | 2.915 | 0.948 | 0.3 [symbol] | 0.973 |

[symbol]

indicates data missing or illegible when filed

As shown in Table 2, the fish oil released from both PCL-ω3 and PCL-ω3-MTKS fibers followed the Korsmeyer-Peppas model. According to this model, the active drug was released from a delivery vehicle in a diffusive manner. In this model, the parameter ‘n’ plays a very important role. The value of ‘n’ determines a type of diffusion mechanism followed by the drug release. If the ‘n’ value is less than 0.45 (n<0.45), the drug release follows Fickian diffusion. In the case of 0.45<n<0.89, diffusion follows the non-Fickian way. In the case of n=0.89 and n>0.89, the drug diffusion follows case I and super case II transport mechanism. In the present disclosure, since the ‘n’ values are less than 0.45 for both 1 and 2, fish oil was released from both fiber matrices (PCL-ω3 and PCL-ω3-MTKS) in a Fickian diffusion way. However, the release of MTKS from PCL-ω3-MTKS showed first-order kinetics, indicating that the release of MTKS tends to depend on a concentration.

### Example 4. In Vivo Animal Study of as-Spun Fibers

An in vivo animal study was performed to confirm a CC reduction effect by the as-spun fibers. Recent studies reported that CC may occur only at 8 to 9 weeks after breast reconstruction/augmentation surgeries. Therefore, an animal study was conducted for 90 days to cause any early symptoms of CC. Animal models were divided into four experimental groups as shown in Table 3 below, and only experimental groups G3 and G4 received mini-implants coated with drug-containing fibers.

TABLE 3

| Animal | No. of | |
|---|---|---|
| group | animals | Treatment |
| G1 | 10 | Mini implant (control) |
| G2 | 10 | PCL fiber + mini-implant |
| G3 | 10 | PCL-ω3 fiber + mini-implant |
| G4 | 10 | PCL-ω3-MTKS fiber + mini-implant |

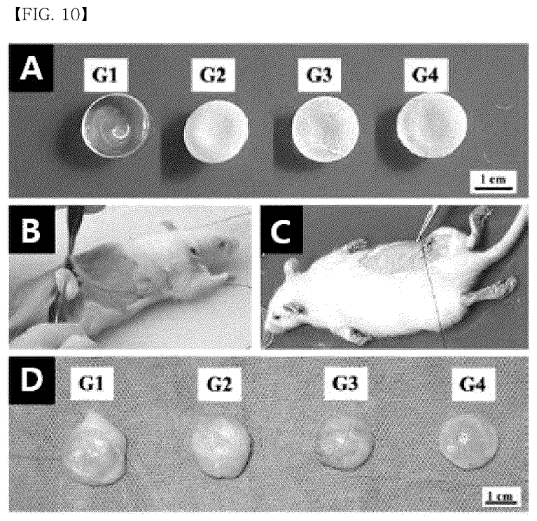

The implants were inserted into the dorsal lumbosacral region of experimental rats, and the animal models were monitored for 90 days ((A) to (C) of FIG. 10). After 90 days, silicone mini-implants and surrounding capsular contracture tissues were harvested ((D) of FIG. 10). Apparently, the experimental group G4 showed the formation of thinner capsular contracture tissues around the mini-implants, which suggested that the PCL/fish oil/MTKS formulation was effective. To get better insights, histological analyses, gene expression studies, and fibrillar collagen SHG imaging were performed with the harvested capsular contracture tissues.

### Example 5. Histological Analyses of Capsular Contracture Tissues

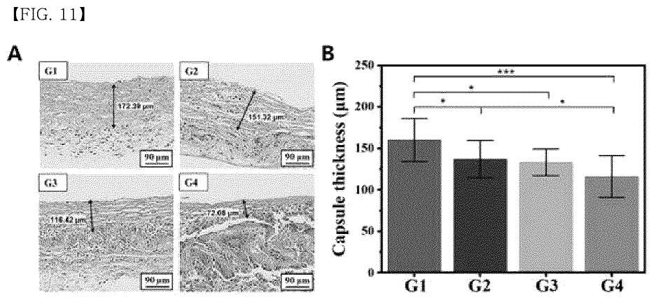

Histological analysis was performed using each removed capsular contracture tissue sample. The tissue morphology and structure were confirmed by H&E staining ((A) of FIG. 11). The average thickness of each capsular contracture tissue was as follows: G1 (160.05±25.95 μm), G2 (136.89±22.76 μm), G3 (133.03±16.12 μm), G4 (115.84±24.97 μm). Compared to G1, the thickness of the capsular contracture tissue significantly decreased in all groups (p<0.05 for G2 and G3; p<0.001 for G4). Particularly, G4 implanted with mini-implants coated with PCL-ω3-MTKS fiber exhibited a significant reduction in the thickness of the capsular contracture tissue compared to G2 (p<0.05). Through this, it was confirmed that the thickness of the capsular contracture tissue was effectively reduced in the case of G2, G3, and G4, which were implanted with mini-implants coated with PCL fiber or drug-encapsulated PCL fiber, and G4 showed the greatest reduction ((B) of FIG. 11). Therefore, it was confirmed that the drug efficacy for preventing capsular contracture formation was demonstrated by ω3, and particularly, the efficacy was increased by a combination of ω3 and MTKS.

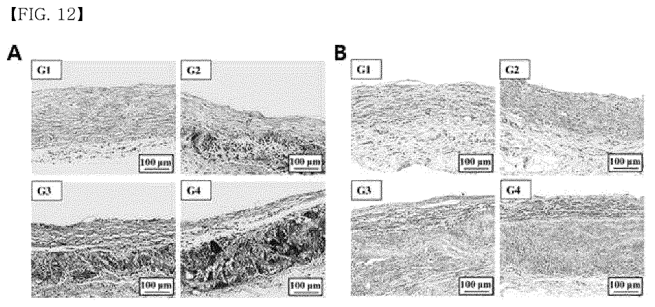

MT staining is a technique used for staining collagen and muscle fibers in a tissue. Only collagen fibers were distinguished in blue to assess fibrosis and compact collagen layers around the implanted mini-implants ((A) of FIG. 12). As a result, it was confirmed that collagen deposition and fibrosis were gradually reduced depending on the experimental group. In G1 as a control group, dense collagen bundles and a compact fibrotic structure were observed, indicating significant fibrosis. G2 showed a moderate reduction in collagen density, whereas G3 exhibited a further decrease with thinner and more loosely packed collagen bundles. In G2 and G3, muscle fibers were stained red, indicating that fibrosis accompanied by structure remodeling was in progress. The most significant reduction of collagen was observed in G4, and the fibrotic layer was the thinnest and the collagen bundles were sparsely distributed. Also, a loose and more porous collagen network means reduced fibrosis with improved biocompatibility in G4.

Next, IHC staining was performed for α-SMA, a key protein in myofibroblasts. Fibrosis includes myofibroblasts that support contractile properties in cells. Thus, an increased amount of α-SMA is associated with fibrotic diseases such as CC. The brown staining for α-SMA indicated a progressive reduction in myofibroblast activity across the groups, whereas weaker staining was observed in normal tissues ((B) of FIG. 12). In G1, α-SMA expression was highest, the staining intensity was strong, and abundant α-SMA positive cells distributed throughout the fibrotic capsular contracture tissues, which indicated that myofibroblasts were significantly activated. In G2, α-SMA staining was moderately reduced compared to that in G1, indicating a partial decrease in myofibroblast activity. G3 confirmed a further reduction in α-SMA expression, with weaker staining and fewer positive cells, confirmed an enhanced antifibrotic effect. Thus, the weaker staining degree in G2 and G3 indicated a transition state with moderate fibrosis. In G4, α-SMA expression was the lowest, the staining intensity was also minimal, and α-SMA positive cells were sparse, indicating significant inhibition of myofibroblast activity with reduced fibrosis. These results were consistent with the measured thickness of the capsular contracture tissue. Taken together, it was confirmed that in G4, implantation of mini-implants coated with PCL-ω3-MTKS fiber most effectively suppressed myofibroblast activity, reduced fibrosis of capsular contracture, and limited collagen deposition.

### Example 6. QRT-PCR Gene Expression Analysis

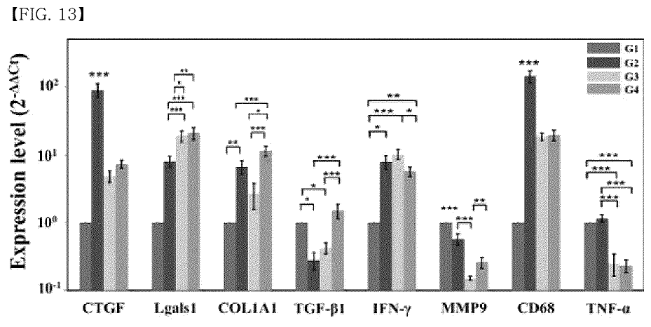

CC involves complex interactions between inflammatory and fibrotic pathways. To quantitatively analyze a correlation between fibrosis-related gene expression and the fibrotic response and inflammation, qRT-PCR was performed on the harvested capsular contracture tissue, and then specific genes such as key markers of fibrosis and inflammation were analyzed. As shown in FIG. 13, Lgals1 expression significantly increased in G3 (18.65±3.55) and G4 (20.44±4.14) compared to G1 (1.00±0.00) and G2 (8.02±1.55) (p<0.05).

Lgals1 (Galectin-1) is known to regulate immune responses and fibrosis by influencing T-cell apoptosis, macrophage polarization, and extracellular matrix remodeling. In G4, it was confirmed that the Galectin-1 was upregulated, so that tissue remodeling was regulated, and fibrosis activity was reduced. Galectin-1, not a direct anti-fibrotic agent, appears to suppress inflammation and promote M2 macrophage polarization, which was estimated to contribute to the reduced fibrosis observed in G4. These results suggest that Galectin-1 indirectly supports balanced tissue remodeling and limits excessive fibrotic responses to highlight its potential as a therapeutic target in fibrosis regulation. TNF-levels also significantly decreased in G3 (0.25±0.09) and G4 (0.23±0.05). In addition, compared to G1 (1.00±0.00) and G2 (1.15±0.15) (p<0.05), G4 indicated effective suppression of inflammatory cytokines, which were key causes of chronic inflammation and fibrosis.

The expression levels of CTGF and CD68 were significantly elevated in G2 (89.08±19.84 and 141.23±29.59, respectively) compared to all other groups (p<0.001). CTGF, a profibrotic marker, promotes fibroblast activation and collagen synthesis and acts as a downstream mediator of TGF-β to amplify fibrotic effects. CD68, expressed by monocyte-lineage cells, is associated with macrophage infiltration, chronic inflammation, and giant cell reactions, and is linked to capsular contracture. These results suggested that G2, in which only the PCL fibers were implanted without drug loading, had exacerbated inflammatory and fibrotic responses. The elevated levels of CTGF and CD68 indicate that the physical presence of PCL fibers alone could potentially contribute to local irritation and macrophage infiltration, which suggests the possibility that unmodified PCL fibers might influence tissue reactions in the absence of anti-inflammatory or modulatory agents.

Interestingly, TGF-β1 expression significantly decreased in G2 (0.28±0.08) and G3 (0.42±0.08) compared to G1 (1.00±0.00) (p<0.05), while G4 (1.50±0.37) showed a significant increase compared to G2 and G3 (p<0.001). TGF-β1, a critical profibrotic cytokine, plays a pivotal role in the development of fibrosis by stimulating fibroblast activation and promoting collagen deposition. TGF-β1 differentiates fibroblasts into α-SMA-positive myofibroblasts, and the myofibroblasts increase collagen synthesis and contractile activity, leading to the thickening and stiffening of the tissues surrounding implants to cause capsular contracture.

However, it was previously confirmed that in G4, TGF-β1 expression was increased, but the thickness of the capsular contracture tissue was significantly reduced. This result suggests that in G4, the components of the treatment, such as fish oil, PCL fiber, and MTKS, acted synergistically to mitigate the profibrotic influence of TGF-β1. The anti-inflammatory properties of the fish oil were combined with its ability to reduce MMP9 expression to be likely to contribute to stabilizing the extracellular matrix and limiting fibrosis. A result of having a high level of TGF-β1 as a key factor of fibrosis in G4 may reflect a controlled repair process rather than pathological fibrosis. The dual role, including anti-inflammatory effects, highlights its complex involvement in balancing tissue repair and fibrosis in this treatment. COL1A1 expression was highest in G4 (11.20±1.77), and significantly increased compared to G1 (1.00±0.00), G2 (6.69±1.53), and G3 (2.68±1.13) (p<0.01). Upregulation of COL1A1 reflects increased collagen deposition, which was a critical factor in the progress of capsular contracture. However, excessive fibrosis in G4 was not shown, which suggests balanced collagen regulation so as to stabilize a tissue structure.

The organization and density of collagen fibers are critical. Disorganized or excessive collagen deposition may lead to capsular contracture formation, while a balanced collagen matrix prevents capsular contracture formation and supports structural integrity. MMP9 levels were significantly reduced in G2 (0.57±0.11), G3 (0.15±0.01), and G4 (0.26±0.05) compared to G1 (1.00±0.00) (p<0.001). As MMP plays a key role in extracellular matrix (ECM) degradation and remodeling, increased MMP-9 expression has been related to capsular contracture.

The further reductions in MMP-9 levels in G3 and G4 compared to G2 indicate enhanced suppression of ECM remodeling, which is likely to contribute to the reduced thickness of capsular contracture tissue observed in G3 and G4. Lastly, IFN-γ expression significantly increased in all experimental groups compared to G1 (1.00±0.00), as confirmed as G2 (7.88±1.86), G3 (10.19±1.72), and G4 (5.68±0.94). As a key cytokine, IFN-γ plays a pivotal role in modulating immune responses, activating macrophages, and inhibiting fibroblast activity, and accordingly, regulates collagen deposition and ECM remodeling. The low level (p<0.05) was shown in G4 compared to G3, thereby suggesting a more balanced inflammatory response in G4, and minimizing excessive inflammation while facilitating controlled tissue repair and remodeling processes. In summary, the experimental group implanted with mini-implants coated with PCL-ω3-MTKS fiber showed significantly lower expression of fibrosis-related genes than the control group, which indicated a potential reduction in capsular contracture severity.

### Example 7. SHG Imaging Study

In the capsular contracture formation, the fibrosis may be classified into three stages: early stage, transition stage, and late stage. A well-regulated macrophage differentiation process contributes to the rapid transition from early to late stages of fibrosis formation, accompanied by mild inflammatory responses. Previous studies have demonstrated that early polarization of macrophages from an inflammatory M1 phenotype to a tissue repairing M2 phenotype plays an important role to lower chronic inflammation and fibrotic tissue deposition. This theory may be supported by a relationship between collagen types, tissue, and fibrosis stage. In the early stage of fibrosis, collagen type III predominates, and has an anisotropic (aligned) structure. Anisotropic collagen fibers contribute to the generation of thick capsular contracture, greater fibrosis, and an increased risk of scarring due to fibroblast activation. As fibrosis advances, type III collagen transforms into type I collagen, resulting in a heterogeneous organization, and collagen remodeling begins and fiber alignment is reduced as the ECM stabilizes. In the late stage, type I collagen dominates with an isotropic (random) organization, resulting in thin and soft capsules with low fibrosis. This represents the end of the ECM remodeling process and improved biocompatibility.

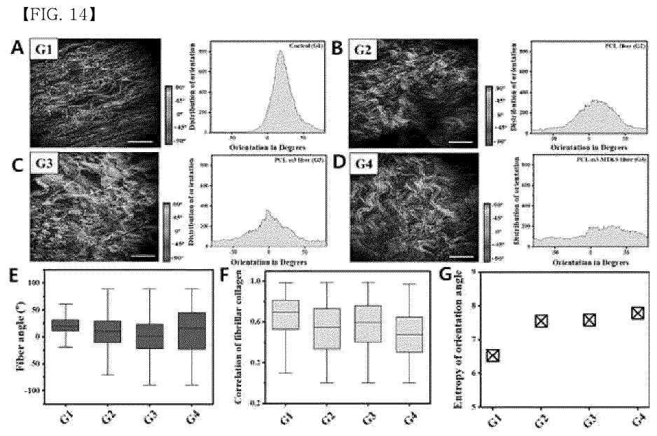

Based on these results, the organization and alignment of the collagen fibers were analyzed by SHG imaging ((A) to (D) of FIG. 14). G1 showed highly organized, aligned collagen fibers, indicating severe fibrosis. According to the colors of fibers in the SHG image, the orientation angle of each fiber may be statistically analyzed. As the analysis result, histogram plots of collagen orientation showed a sharp anisotropic distribution, exhibiting signs of severe capsular contracture. In G2, G3, and G4, the degree of anisotropy was much lower. Among these groups, G2 showed anisotropic collagen orientation indicating signs of capsular contracture. This result is consistent with the high CTGF and CD68 expression levels of G1 and G2, confirmed from qRT-PCR study. G3 showed almost similar collagen fiber distribution to G2. G4 showed relatively the least collagen density among all groups. The collagen fibers of G4 showed an isotropic distribution, which indicated minimal fibrosis similar to healthy tissues. This result is matched with the increased TGF-β1 value confirmed from conventional gene analysis, indicating structured collagen and healthy ECM rather than fibrosis. The fiber angles were confirmed as shown in (E) of FIG. 14 using boxplot. As a result, G4 showed a wider interquartile range (IQR), which indicated isotropy of collagen fibers having a broader spectrum of fiber angles. In addition, G1 showed a narrow IQR and thus showed the highest anisotropy among all experimental groups with collagen fibers angles being aligned towards the median. G2 and G3 had a narrower IQR and a wider whisker than G4, and thus showed a moderate degree of fibrosis. In addition, the entropy of orientation angles of G1, G2, G3, and G4 were compared and shown in (G) of FIG. 14.

The highest entropy confirmed in G4 correlates with the lowest anisotropy. As the collagen fibers became isotropic, the entropy increased with the highest in G4. As the result described as the collagen fiber orientation data, it could be concluded that ω3 and MTKS as active drugs in G4 helped to proceed to the early stages of fibrosis rapidly and to reach the late stage of fibrosis. As a result, it was confirmed that the reason was that the G4 experimental group showed a soft and thin capsule to have the highest biocompatibility. In contrast, G1 without implanted PCL fiber and drugs showed signs of prolonged early-stage fibrosis having excess anisotropic type I collagen fibers (Table 4). As a result, the effect of G4 in reduction of capsular contracture was shown, and it was determined because the drugs, 03 and MTKS, were efficiently delivered from the as-spun fibers.

TABLE 4

| Experimental Group | Stage | Organization | Symptom |
|---|---|---|---|
| G1 | Early | Anisotropic | Thick capsule, high |
| | | | fibrosis |
| G2, G3 | Transition | Mixed | Remodeling |
| G4 | Late | Isotropic | Thin, soft capsule, |
| | | | less fibrosis |

As described above, the specific exemplary embodiments of the present disclosure have been described in detail, but those skilled in the art understanding the spirit of the present disclosure will be able to easily propose other degenerate inventions or other exemplary embodiments included in the scope of the present disclosure by adding, changing, and deleting other elements within the same technical scope. Therefore, it should be appreciated that the exemplary embodiments described above are illustrative in all aspects and are not restricted. The scope of the present disclosure is represented by claims to be described below rather than the detailed description, and it is to be interpreted that the meaning and scope of the claims and all the changes or modified forms derived from the equivalents thereof come within the scope of the present disclosure.

What is claimed is:

1. Electrospun fibers comprising fish oil and a leukotriene receptor antagonist.

2. The electrospun fibers of claim 1, wherein the electrospun fibers are at least one selected from the group consisting of poly(ε-caprolactone) (PCL), polyvinyl alcohol (PVA), polystyrene (PS), poly(lactic-co-glycolic) acid (PLGA), and poly(l-lactic acid) (PLLA).

3. The electrospun fibers of claim 1, wherein the fish oil includes omega-3 polyunsaturated fatty acids.

4. The electrospun fibers of claim 1, wherein the leukotriene receptor antagonist is montelukast or a pharmaceutically acceptable salt thereof.

5. The electrospun fibers of claim 1, wherein the electrospun fibers have an average diameter of 1 to 3 μm.

6. The electrospun fibers of claim 1, wherein the electrospun fibers have a water contact angle of 40.81±1.63.

7. A composition for implantation comprising electrospun fibers containing fish oil and a leukotriene receptor antagonist.

8. The composition for implantation of claim 7, wherein the composition for implantation has an effect of preventing, improving or treating capsular contracture.

9. A method for preparing electrospun fibers containing fish oil and a leukotriene receptor antagonist, the method comprising:
dissolving electrospun fibers in a solvent;
preparing a first mixture by mixing fish oil into the solvent in which the electrospun fibers are dissolved;
preparing a second mixture by adding a leukotriene receptor antagonist to the first mixture; and
spinning the second mixture using an electrospinner.

10. The method for preparing the electrospun fibers of claim 9, wherein the electrospun fibers are at least one selected from the group consisting of poly(ε-caprolactone) (PCL), polyvinyl alcohol (PVA), polystyrene (PS), poly(lactic-co-glycolic) acid (PLGA), and poly(l-lactic acid) (PLLA).

11. The method for preparing the electrospun fibers of claim 9, wherein the fish oil includes omega-3 polyunsaturated fatty acids.

12. The method for preparing the electrospun fibers of claim 9, wherein the leukotriene receptor antagonist is montelukast or a pharmaceutically acceptable salt thereof.

13. The method for preparing the electrospun fibers of claim 12, wherein the pharmaceutically acceptable salt of montelukast is montelukast sodium.

14. The method for preparing the electrospun fibers of claim 9, wherein the solvent is a mixed solvent of chloroform and methanol.

15. The method for preparing the electrospun fibers of claim 14, wherein the chloroform and methanol is mixed in % v/v of 1 to 2:1.

16. The method for preparing the electrospun fibers of claim 9, wherein the electrospun fibers are included in an amount of 11 to 13% (w/v).

17. The method for preparing the electrospun fibers of claim 9, wherein the fish oil is included in an amount of 25 to 55% (w/v).

18. The method for preparing the electrospun fibers of claim 9, wherein the leukotriene receptor antagonist is included in an amount of 0.5 to 4% (w/v).

19. The method for preparing the electrospun fibers of claim 9, wherein the electrospun fibers are spun under conditions of a feed rate of 2 to 5 ml/hr and an applied voltage of 10 to 30 kV.

20. A method for preventing, improving or treating capsular contracture in a subject in need thereof, the method comprising implanting into an implantation site of the subject a composition comprising the electrospun fibers according to claim 1.


SEQUENCE LISTING (18 sequences)

SEQ ID NO: 1 - DNA, 22 bp, Synthetic construct
  source 1..22: mol_type = other DNA
catcatgaagtgtgacgttgac

SEQ ID NO: 2 - DNA, 19 bp, Synthetic construct
  source 1..19: mol_type = other DNA
gcatcctgtcagcaatgcc

SEQ ID NO: 3 - DNA, 18 bp, Synthetic construct
  source 1..18: mol_type = other DNA
tgcaacagcaaggacggc

SEQ ID NO: 4 - DNA, 19 bp, Synthetic construct
  source 1..19: mol_type = other DNA
cacctctgcaacacttcca

SEQ ID NO: 5 - DNA, 23 bp, Synthetic construct
  source 1..23: mol_type = other DNA
atagttcaccacggaaacacctg

SEQ ID NO: 6 - DNA, 22 bp, Synthetic construct
  source 1..22: mol_type = other DNA
gcagcaaccttctcctttaatg

SEQ ID NO: 7 - DNA, 19 bp, Synthetic construct
  source 1..19: mol_type = other DNA
cccagcatctgcaaagctc

SEQ ID NO: 8 - DNA, 20 bp, Synthetic construct
  source 1..20: mol_type = other DNA
gtcaatgtacagctgccgca

SEQ ID NO: 9 - DNA, 20 bp, Synthetic construct
  source 1..20: mol_type = other DNA
gagcggagagtactggatcg

SEQ ID NO: 10 - DNA, 20 bp, Synthetic construct
  source 1..20: mol_type = other DNA
gcttcttttccttggggttc

SEQ ID NO: 11 - DNA, 21 bp, Synthetic construct
  source 1..21: mol_type = other DNA
aggtgcctcggatggttatcg

SEQ ID NO: 12 - DNA, 20 bp, Synthetic construct
  source 1..20: mol_type = other DNA
tgcttgcccaggaagacgaa

SEQ ID NO: 13 - DNA, 23 bp, Synthetic construct
  source 1..23: mol_type = other DNA
cgaaaagctgactaattattcgg

SEQ ID NO: 14 - DNA, 20 bp, Synthetic construct
  source 1..20: mol_type = other DNA
ctcttcgacctcgaaacagc

SEQ ID NO: 15 - DNA, 21 bp, Synthetic construct
  source 1..21: mol_type = other DNA
aagcctgtagcccacgtcgta

SEQ ID NO: 16 - DNA, 24 bp, Synthetic construct
  source 1..24: mol_type = other DNA
ggcaccactagttggttgtctttg

SEQ ID NO: 17 - DNA, 19 bp, Synthetic construct
  source 1..19: mol_type = other DNA
tgttgcggaaatacaagca

SEQ ID NO: 18 - DNA, 20 bp, Synthetic construct
  source 1..20: mol_type = other DNA
ggcagcaagagagattggtc